(19) United States
(12) Patent Application Publication
(10) Pub. No.: US 2004/0219190 A1
(43) Pub. Date: Nov. 4, 2004

(54) Multi-layer transenamel bleaching system

(76) Inventor: Carl Kosti, Mason, OH (US)

Correspondence Address: R. William Graham, 22 S. St. Clair St., Dayton, OH, 45402, US

(21) Appl. No.: 10/427,123

(22) Filed: May 1, 2003

(51) Int. Cl. A61K 7/20; A61K 7/135; A61K 9/70

(52) U.S. Cl. 424/449; 424/62

(57) ABSTRACT

A sustained release transenamel bleach delivery oral composition having built-in hydroxyl ions—hydrogen ions exchange capability for topical application on teeth in an oral cavity.

### FIELD OF THE INVENTION

This invention relates to improved dental transenamel bleach delivery system and more particularly to a multi-layered, semi-rigid laminate system that has built-in exchange of hydroxyl ions—hydrogen ions capability to provide topical delivery of selected active agents for a sustained period of time following its application to plurality of enamel surfaces comprising (1) a bleach-stable carrier of active substances film laminate said film laminate may comprise of (a) one outer water and moisture impermeable layer and (b) one inner moisture and saliva permeable and swelling alkaline film sublayer of bleach-stable material, preferably polymer, complexed polymer or copolymer, acting as a latent hydroxyl ions donor and having the pH between about 6.5 and 10.5 and present in the operative range of about 2.5 percent and 75 percent with respect to the total weight of the composition and (2) a hydrophilic bleach-stable film layer acting as a hydroxyl ions receptor and having the pH between about 4.5 and 6.8 said layer consisting of organic or inorganic substances present in the operative range of about 2.5 percent and 75 percent with respect to the total weight of the composition and wherein dispersed, emulsed, dissolved, suspended or otherwise incorporated throughout said layer is an effective amount of one or more peroxygens present in an operative range of about 1.5 percent and 55 percent with respect to the total weight of the composition and the said rigid to semi-rigid transenamel system when exposed to saliva and oral moisture will transform into a soft and flexible unit that easily conforms to the teeth contour.

The system is “freeze-dried” by evaporation of most liquid solvents thus immobilizing the peroxygen and prevents said acid peroxygen from migrating into the alkaline layer, the said dried rigid or semi-rigid film is then cut into desired dimensions, usually from about 0.5 cm wide by 12 cm long and 0.5 mm thick. The film, or tape, when exposed to the saliva and moisture in the oral cavity becomes soft and flexible which can simply be topically applied and aggressively adhered to selected plurality of tooth surfaces for desired effect, such as bleaching , and allowed to remain in position for about between 30 and 60 minutes, without adhering to, or otherwise chemically affecting, the mucous membranes and other oral soft tissues The water soluble phase of the complexed transenamel system film comprising the dried peroxygen, which, during its intended function in the oral cavity, dissolves at a faster rate to provide freshly dissolved peroxygen to generate quick onset of whitening effect on the enamel surfaces. The alkaline moisture and saliva permeable portion of the film slowly activates the latent hydroxyl ions to raise the pH of the water soluble film layer to approximately between 7.5 and 8.5 thus enhancing the activity of the latent peroxygen. The insoluble film phase can readily be removed by stripping the film from treated tooth surfaces.

### THE PRIOR ART

It has long been recognized by the dental professionals that hydrogen peroxide and other peroxygen agents are effective in curative and cosmetic treatments with respect to soft oral tissues diseases, such as periodontal disease, and bleaching of tooth surfaces. Prior to early 1980's most tooth bleaching procedures were conducted by the in-office method directly by the dentists and dental hygienists. This method was time consuming and very expensive. Since late 1980's new developments in methodology and products for home-use bleaching of teeth became popular with dentists and patients alike. The technique included: taking an impression of the patient's mouth using a rubberized material; reproducing a stone or plaster cast of the impression; forming a tray of the patient's dentition using a thermoforming material and then applying an amount of the bleaching gel into each reservoir of the teeth to be bleached and finally, placing the filled tray into the mouth to cover the teeth completely. The bleaching gel composition was to be replenished every one to 2.5 hours due to the loss of the gel over the edges of the tray. This undesired flow of the bleaching gel composition may lead to accidental swallowing and subsequent systemic complications. Retaining Of the bleaching tray in the mouth during the night was not only uncomfortable but also dangerous.

It is also long been recognized by the dental professionals that hydrogen peroxide and other peroxygens are most effective when prepared fresh just prior to being employed in the oral cavity for whitening or bleaching.

The most commonly used bleach agents are hydrogen peroxide and carbamide peroxide. Carbamide peroxide is also known in the art as urea peroxide, hydrogen peroxide carbamide and perhydrol urea. Varieties of gel viscosities have been introduced in the market for bleaching of teeth with mixed results. It is well known that the efficacy of the bleaching procedure depends on many factors including the intensity and type of stains, duration of the bleaching procedure, mechanism of formulating and compounding of the composition to provide sufficient available bleaching active ingredients on the tooth surfaces, and finally the natural capacity of the enamel for bleach up-take. Although home use method of bleaching teeth is less expensive than the in-office procedure it has serious disadvantages in that the bleaching gel composition must be replenished frequently during usage and treatment may last for several weeks or months. However, the most serious disadvantage of the home-use bleaching technique is the inexperience of the user in expressing the proper amount of the bleaching gel composition into the tray. Voluminous studies have shown that swallowing of the gel bleaching composition and saliva dilution factor diminish the amount of the active ingredient in the bleaching tray. Test results have indicated that only less than half of the original bleaching composition is present in the tray after one hour of treatment. The side effects of repeatedly ingesting large amounts of bleaching agents range widely from local irritation and chemical bums of mucous membranes, general periodontal disease to chronic exposure that may lead to tissue mutation and cancerous growth. Therefore, it is essential that the amount and frequency of exposing the oral soft tissues to bleaching agents be kept to a minimum. Today, almost exclusively peroxygens such as hydrogen peroxide and carbamide peroxide are used in liquid or gel form. I am not aware of any suggestion in literature proposing the employment of a dry form of a peroxygen at the time of use.

Dental trays are very inconvenient for the wearer and are not favorably received by almost all patients thus making the compliance of the treatment regimen difficult to maintain.

U.S. Pat. No. 4,645,662 to Nakashimi et al. discloses an oral gel composition for treatment of dental hypersensitivity consisting of aluminum and a carboxylate compound such as carboxyethyl cellulose or carboxypropyl cellulose. However, elemental aluminum, being a transition metal, reacts readily with peroxygens to almost render the compound totally ineffective.

U.S. Pat. No. 4,713,243 to Schiraldi et al. discloses an extruded film which is bio-adhesive for intra-oral controlled release delivery of medicament comprising suitable active agent and hydroxypropyl cellulose and ethylene oxide homopolymer. This patent makes no suggestion of dried multi-layered transenamel systems for prolonged topical application in the oral cavity comprising hydrophilic and hydrophobic components.

U.S. Pat. No. 4,315,779 to Heyd et al. discloses non adhesive denture retention gel composition comprising inter alia, a hydrophobic cellulose polymer, such as hydroxypropyl cellulose.

U.S. Pat. No. 5,425,953 to Sintov et al. discloses a film forming liquid polymer composition comprising a water soluble cellulose polymer, for example hydroxypropyl cellulose, hydroxypropyl ethylcellulose, hydroxypropyl methylcellulose and carboxymethylcellulose; a peroxy compound; a stabilizing additive for the peroxy compound; and a vehicle consisting of ethyl alcohol, and ethyl alcohol and water. Again, this patent does not suggest dried hydrophilic and hydrophobic multi-layered transenamel adhesive system for topical application on tooth surfaces for a selected sustained release function of long duration.

U.S. Pat. No. 5,438,076 to Friedman et al. discloses methods for treatment of gingivitis, oral plaque and oral and dermatological fungal infections by administration of a liquid methacrylic acid copolymer composition containing a release adjusting agent and a pharmacological agent. More particularly, Friedman relates to a method for removing or inhibiting of oral plaque build-up-up and for treatment of gingivitis comprising a liquid polymer composition consisting one or more sustained release methacrylic acid polymer; a bactericidal pharmacological agent, such as an antibiotic and an antiseptic; a release adjusting agent capable of cross-linking , agent such as citric acid, sodium citrate, sodium diocusate and an amino acid; and a vehicle consisting of methacrylic acid type A copolymer or combination of methacrylic acid and methacrylate, or a methacrylic acid type B or a combination of methacrylic acid and methyl methacrylate; or a dimethyl animoethyl acrylate/ethylmethacrylate copolymer having a low quaternary ammonium groups, and/or an ethylmethacrylate/chlorotrimethyl ammoniumethyl methacrylate copolymer having a low content of quaternary ammonium groups. It is well known in the art that methacrylic acid and methylmethacrylate are extremely irritating to eyes, skin and mucous membranes and are not practical for topical application in the oral cavity.

U.S. Pat. No. 5,575,654 issues to Fontenot discloses a prepackaged moldable dental appliance, adopted to fit variously sized dental arches which contains a premeasured amount of medicinal or bleaching agents. Prior to use the appliance is removed from the packaging, aligned to the edges of the teeth and pushed over the teeth in the direction of the periodontal tissue until it covers teeth surfaces.

U.S. Pat. No. 5,310,563, issued to Curtis et al. discloses a putty-like material which is formed by pressing against the teeth. The composition encapsulates the active bleaching elements while the putty is held in position by the undercut surfaces of the teeth.

U.S. Pat. No. 5,279,816 issued to Church et al. discloses an oral gel composition effective in whitening tooth surfaces comprising an amount of peroxyacetic acid.

U.S. Pat. No. 5,425, 953 issued to Sintov et al. discloses storage stable liquid polymer, oral composition containing a water soluble, low viscosity, film forming cellulosic polymer, bleaching agent and vehicle U.S. Pat. Nos. 5,879,691 and 5,891,453 issued to Sagel et.al. disclose a two layered dental bleaching strip comprising water insoluble film support coated with an active gel. The bleaching actives incorporated in a water soluble carboxypolymethylene gel composition in one layer coated on the second layer that is clear, flexible water insoluble, water impermeable polyethylene film support. The bleaching elements of the system of this invention is already active even prior to its topical application on plurality of tooth surfaces and does not undergo through any chemical interaction between the two layers. Its chemical activity is predetermined during manufacturing process without any built-in hydroxyl-hydrogen ions exchange when wetted in the oral cavity.

U.S. Pat. No. 6,083,421 issued to Huang et al. discloses a tooth whitening varnish composition comprising carbamide peroxide, film forming water insoluble agent and volatile organic solvent. The film forming agent is selected from cellulose, polyvinyl, butyral, coumarone resin or shellac.

It is a well known fact in the art that hydrogen peroxide and other peroxygens react very slowly in almost neutral pH and in order to quickly obtain a certain level of color change a high concentrations of the hydrogen peroxide must be used. High concentrations, however, are toxic and can cause untoward side reaction which may be harmful to soft oral tissues, such as chemical burns, and to the dental pulp resulting in pulpits, or even periapical abscess.

The present invention overcomes these and other problems and disadvantages of the prior art by providing transenamel bleaching system comprising one or more bleach-stable hydrophilic and hydrophobic phases of a film forming materials, preferably polymers, copolymers or crosspolymers, wherein one phase is a donor and comprises latent hydroxyl ions and another phase is an receptor and comprises latent hydrogen ions the said system prior to its usage is in relatively dry state and inert which can be selectively and topically applied to plurality of tooth surfaces and in presence of saliva or moisture activates the chemical hydroxyl-hydrogen ions exchange to substantially raise the systems pH and thus affect a desired bleaching effect in a predetermined time period before dissolution in the oral cavity by the saliva. The present improvement in tooth bleaching system allows for employment of smaller amounts of the bleaching actives due to its built-in higher pH. It comprises actives, such as peroxygens, dissolved, emulsed, distributed or otherwise incorporated throughout a hydrophilic, semi-hard, pseudoplastic, flexible, adhesive film having the characteristics to stand alone without additional support, or substrate, and a hydrophobic laminate layer comprising one water and saliva impermeable layer and another moisture permeable alkaline layer. Such film is introduced to a low-heat, moisture free environment to evaporate the liquid solvents of the composition and form a “freeze-dried” film wherein the peroxygen is in a relatively dry form and distributed or dispersed throughout said film that when in contact with plurality of tooth surfaces, preferably wet surfaces, will adhere tenaciously to said tooth surfaces to produce freshly dissolved peroxygen to provide bleaching effect in a short period of time without affecting other hard tissues and mucous membranes. The differences will become more fully understood by the illustrations provided in the following discussion.

### SUMMARY OF THE INVENTION

The present invention deals with a transenamel system having therein built-in hydroxyl-hydrogen ions exchange capability for delivery of bleaching agents directly on plurality of tooth surfaces comprising (1) a bleach-stable carrier of active substances film laminate consisting of (a) one saliva and moisture impermeable layer and (b) one saliva and moisture permeable and swelling alkaline donor sublayer containing latent hydroxyl ions and (2) a hydrophilic, bleach-stable receptor phase, or layer, of bleaching actives the said actives being latent hydrogen ions. The process of making such two-phase film consists of first coating one phase of the invention, generally the saliva and moisture impermeable composition and then casting a sublayer having the pH adjusted between about 6.5 and 10.5 to form the hydrophobic laminate and then, when the moisture permeable alkaline donor layer sets-up, casting upon it the hydrophilic phase comprising the bleaching actives dispersed or distributed throughout the bleach-stable composition comprising organic and inorganic film forming substances and then adjusting the pH of the phase to about between 4.5 and 6.8 The casted viscous film is allowed to air dry at room temperature or in warm air to expel the liquid solvents by evaporation thus producing a dry film that is soft, pseudoplastic, flexible, clear, transparent, pressure sensitive to wet tooth structures that will not adhere to soft oral membranes and wherein the hydroxyl ions and hydrogen ions are in inactive, latent, state which when applied to the selected plurality of teeth, with the hydrophilic phase placed in contact with tooth surfaces, to provide aggressive adhesion to said tooth surfaces, the said dried film when wetted is capable of activating the chemical hydroxyl-hydrogen ions exchange between the two layers and raising the pH of the system to a relatively high level thus providing sustained release bleaching action to the contacted tooth surfaces employing smaller amounts of the bleaching actives without appreciably affecting the mucous membranes and other oral soft tissues. The water and saliva soluble phase of the bleaching film will dissolve slowly to provide continuous bleaching within the predetermined period of time. The undissolved hydrophobic phase can easily be removed by stripping from the enamel surfaces or by brushing.

A “swelling” layer in accordance with the present invention refers to an increase in volume of the sublayer in a predictable manner without forming a gel when exposed to moisture, saliva and water.

A “continuous phase” in the present invention refers to a state wherein usually one or more compounds, either organic or inorganic, are dissolved to form one homogeneous phase.

A “discontinuous phase” refers to a phase composition consisting of two or more compounds, organic or inorganic, wherein at least one compound is hydrophobic and distributed throughout the continuous phase.

An “inclusion complex copolymer” in the present invention refers to a chemical reaction, or state, wherein the rate of desolation of one less soluble polymer may be enhanced by including a more soluble polymer to form a complex having the characteristics different than either one polymer alone.

An “element” or an “active” in the present invention is defined as any chemical active substance which will produce a change or alteration in its structure when exposed to another active substance.

A “freeze-dried” process in the present invention relates to a quick evaporation of the liquid solvents form the composition and rapid hardening of the film to immobilize the solid peroxygen and prevent its dissolution and thus its degradation.

A “hydroxyl-hydrogen ion exchange” in the present invention relates to the expected ionic chemical reaction between an acid and a base wherein a base functions as an hydroxyl ion donor and hydrogen ion receptor and the acid functions as a hydrogen ion donor and hydroxyl ion receptor.

It is an object of the present invention to provide an improved transenamel bleach delivery system for topical application to plurality of tooth surfaces by providing a composition containing therein built-in hydroxyl ion—hydrogen ion exchange capability comprising (1) an effective amount of bleach-stable carrier of active substances film forming laminate material, preferably polymer, copolymer or crosspolymer, present in an operative range of about 2.5 percent to 75 percent with respect to the total weight of the composition and wherein the laminate layer comprises (a) an effective amount of a saliva and moisture impermeable outer layer and (b) an effective amount of a saliva and moisture permeable and swelling alkaline sublayer having the pH adjusted to between about 6.5 and 10.5 and (2) an effective amount of hydrophilic acid layer comprising one or more bleaching actives present in the operative range of about 1.5 percent and 55 percent with respect to the total weight of the composition homogeneously distributed, emulsed, dissolved suspended or otherwise incorporated throughout a bleach-stable film forming blend of organic and inorganic substances, said layer being present in in the operative range from about 2.5 percent and 75 percent with respect to the total weight of the composition and wherein the pH is from about 4.5 and 6.8. The formed transenamel bleach delivery system is “freeze-dried” to a semi-rigid state by subjecting it to room temperature air to remove liquid solvents from their molecules and cut to desired dimension and then topically applied with the hydrophilic phase in contact to the selected plurality of tooth surfaces. Upon exposure to oral saliva and moisture the semi-rigid system transforms into a soft, flexible and swollen film. Finger pressure on the outer aspects of the lips may be applied to contour the film to flow in the interdental spaces to insure throughout whitening effect.

It is yet an object of the present invention to provide an improved transenamel bleaching system wherein the support is a chemical agent and a carrier of active substances.

It is also an object of the present invention to provide an improved transenamel bleaching system comprising at least one layer that is a hydroxyl ions donor and another layer that is hydoxyl ions receptor.

It is furthermore an object of the present invention to provide an improved transenamel bleaching system comprising at least one layer that is hydrogen ion receptor and another layer that is hydrogen ion donor.

It is yet an object of the present invention to provide an improved transenamel whitening system wherein the hydroxyl ions and hydrogen ions are in latent state and activated in presence of moisture or water or saliva.

It is still yet an object of the present improved invention to provide whitening system wherein the pH of the two phases when activated is between 7.5 and 10.5.

It is also an object of the present invention to provide an improved transenamel bleach delivery system wherein the two phases when superimposed an inclusion complex is formed at the junction the inclusion complex having the capacity of sustained action.

It is still further object of the present invention of the type stated wherein the two phases are incorporated into one single, indistinguishable and inseparable unit.

It is further an object of the present invention to provide an improved transenamel bleaching delivery system for topical application to the tooth surfaces that does not employ a gel as vehicle for the bleaching actives.

It is another object of present invention to provide an improved transenamel bleaching system for topical application to selected plurality of tooth surfaces wherein the whitening film is relatively semi-rigid and dry before its employement and soft and flexible when applied in the oral cavity when exposed to the saliva and moisture sufficiently that upon pressure with fingers or lip musculature has the ability to effectively flow into interdental spaces to provide complete whitening effect.

It is further an object of the present invention to provide an improved transenamel bleaching system wherein the whitening film may be flexible, clear and transparent.

It is still yet an object of the present invention to provide a transenamel drug delivery system for topical application to tooth surfaces comprising a rate-controlled membrane, said membrane being water and saliva slowly soluble to allow sustained passage of the alkaline elements into the water soluble inner bleaching layer to enhance the activity of the bleaching elements.

It is further still another object of the present invention to provide an improved whitening system of the type stated wherein the hydrophilic phase comprises at least one inorganic compound with at least one organic compound and resultant complex having characteristics different from that of each compound.

It is another object of the present invention to provide an improved transenamel drug delivery system of the type stated wherein the active agents have minimal effect on the surrounding oral soft tissues.

It is still another object of the present invention to provide an improved transenamel drug delivery system that in addition to the active ingredients may comprise suspended, emulated, dispersed, distributed or dissolved therein auxiliary formulating agents such as water, sweetening agents, flavors, calcium, phosphorus, other minerals, vitamins, alcohol, sweeteners, flavors, preservatives, colorants, adhesives, solvents, moisturizers, tooth desensitizes, surfactants, and similar formulating and therapeutic substances wherein the transenamel system can easily be topically applied, and aggressively adhere to the selected tooth surfaces to provide a steady sustained release of the peroxygen and its up-take by the enamel rods to effect bleaching action, the transenamel system can then readily degrade or be stripped from the tooth surfaces.

Other features and advantages of the present invention will be made more apparent as this description proceeds, reference being had to the accompanying drawings herein.

Such transenamel drug delivery systems for whitening tooth surfaces are disclosed and claimed herein.

### DESCRIPTION OF THE DRAWINGS

Other features and advantages of the present invention will be made more apparent as this description proceeds, reference being had to the accompanying drawings wherein:

FIG. 1 illustrates a cross section of a transenamel bleaching tape constructed in accordance with teachings of this invention showing from top to bottom the saliva and moisture impermeable laminate protective layer, moisture permeable alkaline sublayer, hydrophilic bleaching layer and removable release liner.

FIG. 2 is an enlarged view similar to FIG. 1 showing from top to bottom the protective laminate comprising the water and saliva impermeable outer layer and moisture permeable alkaline sublayer containing therein the latent alkaline elements and activated alkaline elements; the dried hydrophilic layer containing therein the latent acid bleaching elements; the release liner removed showing saliva layer covering dental enamel.

FIG. 3 illustrates the activated alkaline elements of the moisture permeable sublayer migrating into the hydrophilic layer to activate the bleaching actives in the hydrophilic layer and the free oxygen diffusing through the saliva layer into enamel layer.

FIG. 4 illustrates another embodiment of the present invention showing the from top to bottom the saliva and moisture impermeable protective layer; moisture and saliva permeable sublayer with latent alkaline elements, permeable rate control membrane, hydrophilic layer with latent bleaching actives, where the removable release liner has been removed and migration is occurring through the saliva layer into the enamel.

FIG. 5 illustrates the relative solubility of the bleaching and alkaline layers of the tapes, or films, of the tlus invention; and

FIG. 6 illustrates pH variations dllring the bleaching stages of the process of this invention.

### DESCRIPTION OF THE PREFERRED EMBODIMENT

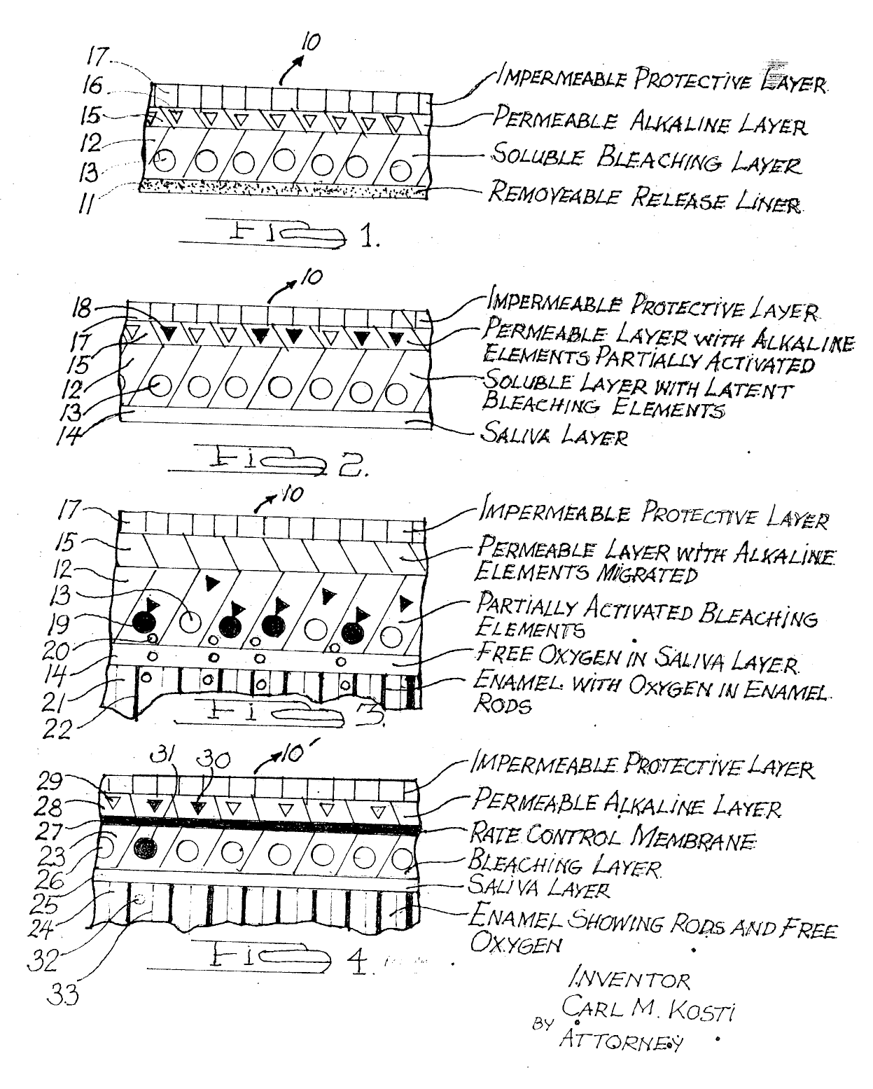

In utilizing a process and bleaching tapes of this invention, and in reference to FIGS. 1 through 3 a transenamel bleaching tape system indicated generally by reference numeral 10 and prior to its application in the oral cavity a protective release liner 11 is removed to expose a hydrophilic layer 12 comprising latent and inactive bleaching actives 13 which is subsequently placed in contact with plurality of selected enamel surfaces. A saliva layer 14 diffuses into the hydrophilic layer 12 and thence into a moisture permeable layer 15 comprising the latent alkaline elements 16. An outer saliva and moisture impermeable layer 17 prevents the escape of the saliva and moisture outside the oral cavity to create swelling effect thus building the osmotic pressure within layer 15 to convert some of the latent alkaline elements 16 into hydroxyl ions 18, as seen in FIG. 2. Due to the increased osmotic pressure in the alkaline layer 15 created by the impermeable protective layer 17 and the hydrophilic layer 12, the active hydroxyl ions 18 are forced by reverse osmosis into the hydrophilic layer 12 where they convert the latent bleaching elements 13 into active bleaching elements 19, as seen in FIG. 3. The diffusion of the hydroxyl ions 18 initially occurs in the most proximal areas in the hydrophilic layer 12 and raises the pH in that particular area to about between 8.5 and 10.5 to activate the latent bleaching actives 13. The activated bleaching elements 19 generate free oxygen 20 which enters the saliva layer 14 and thence readily diffuses into the enamel surface 21 and enamel rods 22. During the bleaching process the water impermeable hydrophobic layer 17 remains unaffected. The conversion of the latent bleaching actives 13 to generate free oxygen 20 is not instantaneous but it is sequential. The latent bleaching actives 13 closest to the alkaline layer 15 are activated first and then the next layer in the hydrophilic layer 12 containing the latent bleaching actives 13 is activated to generate free oxygen 20 and the process continues until most of the hydrophilic layer 12 is dissolved. The entire process of activating the bleaching actives 13 starts in about 30 seconds and it may lasts for approximately more than 1 hour. The undissolved hydrophilic layer 12, the residual alkaline layer 15 and the hydrophobic layer 17 may then be removed from the enamel surfaces and discarded.

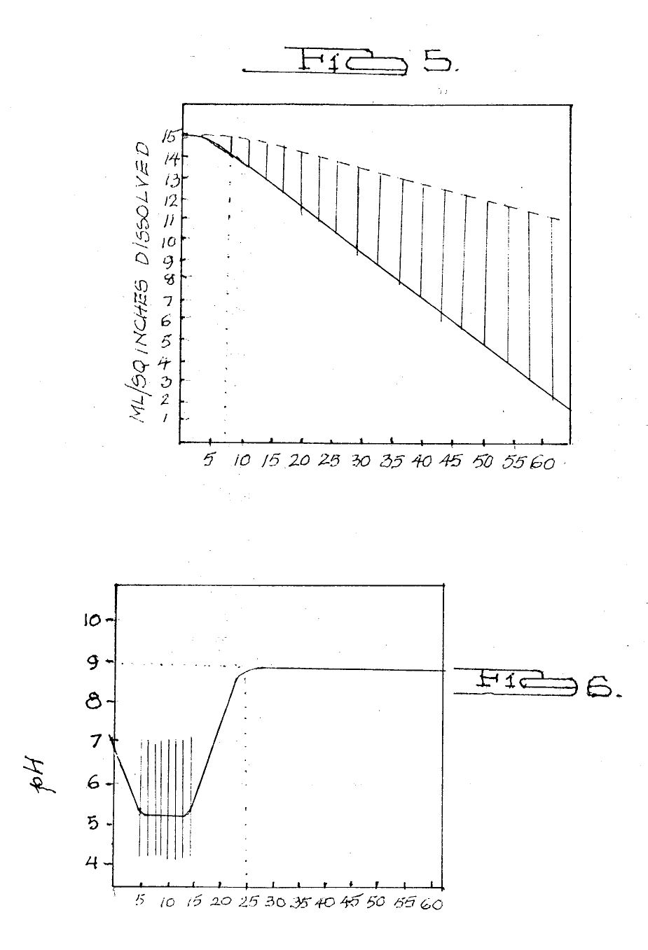

Another method of making and utilizing the process and bleaching tapes of this invention, is generally seen in reference to FIG. 4 Here, a transenamel bleaching tape system is indicated generally by reference numeral 10. Removal of a protective release liner exposes a hydrophilic layer 23 which is topically applied to enamel 24 of plurality of teeth and is exposed to-saliva layer 25 on the surface of tooth enamel 24. The saliva layer 25 diffuses into a hydrophilic layer 24 comprising latent bleaching elements 26 and without affecting latent bleaching actives 26 and diffuses through a rate control membrane 27 into a hydrophobic moisture permeable alkaline layer 28 to dissolve latent alkaline elements 29 and convert them into hydroxyl ions 30 which then by reverse osmosis diffuse through the rate control membrane 27 into the hydrophilic layer 23 to activate the latent bleaching elements 26 into active bleaching elements 31 which readily generate free oxygen 32 and diffuse through the saliva layer 25 into the enamel 24 and thence into the enamel rods 33 to affect bleaching.

The transenamel system may be formulated in such a way that it may be formed to the contour of the tooth surfaces and retain the contour without changing its shape in any way. This can be accomplished by having flexible layers, preferably the occlusive hydrophobic backing, which, although pliable, has no elastic characteristics.

Further, the transenamel system may be formed to cover the entire tooth surface; facial and lingual, or it may be of such design to cover only the facial surfaces. It may also be designed in such manner that the system is contoured at the gingival margin to contact the entire tooth without adversely affecting the gingivae that may cause untoward chemical burns.

To demonstrate what effect osmotic pressure has upon the permeable alkaline sublayer of the laminate layer in activating the latent bleach elements of the transenamnel bleaching system of the present invention several test samples were prepared and then subjected into beakers containing 10 ml water. by the following methods, as examples:

Method I. 1. In a 500 ml glass beaker, 50 grams of polyethylene was dissolved in 100 ml. Acetone with gentle stirring until a viscous composition is obtained. The viscosity of the active composition is between about 500,000 and 1,500,000 cps.

The viscous saliva and moisture impermeable composition was casted on glass surface measuring 12 inches by 16 inches at a rate of about 1 gram/cm2 and spread from end to end using plastic spatula to produce a film 0.5 mm thick. The film was allowed to dry quickly to a semi-hard consistency. It was sticky when touched. 2. In a separate beaker, 10 grams of film forming, hydrophobic and saliva and moisture permeable polyvinylpolypyrrolidone (PVPP, Crospovidone M, BASF) was added in 100 ml of warm water that has been neutralized with 10% sodium hydroxide to a pH of about 8.5 and 10.5. Then 10 ml of pH indicator phenolphthalein was added until a pink discolorations appeared throughout the composition. Violent stirring was avoided to prevent bubble formation. The viscosity of the composition is between about 500,000 and 1,500,000 cps.

This viscous saliva and moisture permeable and swelling composition was casted on top of the still sticky polyethylene film at the same rate and in the same manner as the polyethylene film in the step I above. The total thickness of the water impermeable and saliva and moisture permeable and swelling polymer laminate is about 1 mm. The solvents were quickly evaporate to produce a water insoluble but moisture and saliva slowly permeable dry film laminate. 3. In a separate glass beaker 15.0 grams of vegetal polymers containing xanthan gum, glucose and carrageenan (Lipomoist 2013/Centerchem, Inc.) and 15.0 grams of polyvinylpyrrolidone (PVP K-90/ISP) were dissolved in 100 ml purified water that was neutralized with 2 grams sodium hydroxide to a pH of about 6.8 with constant stirring until the polymers are completely in solution. To this complex mixture 20 mls of hydrogen peroxide were added and gently stirred to provide homogeneous dispersion before 10 ml. of pH indicator bromophenol red was added with gentle stirring to produce a yellow dispersion. When in solution the viscosity of the dispersion is between about 500,000 and 1,5000,000 cps.

Before the composition in 1 and 2 begins to set-up, but it is still tacky to touch, the dispersion in 3 was casted upon the hydrophobic polymer laminate composition 2 at a rate of 2 grams/cm₂ and spread from end to end using a spatula to produce a second film of approximately 1.0 mm thickness and dispersion 3 blends into composition 2 at the juncture to produce an inclusion complex less soluble than the hydrophilic phase but more soluble than the hydrophobic phase. The total thickness of 1, 2 and 3 was 2 mm. 4. The casted film is then immediately dried at low heat to remove the liquid solvents and prevent migration of hydrogen peroxide, which remains largely in crystalline state, into the alkaline sublayer. The dried film is then cut to the desired measurements and shapes. The dimensions of each tape film is preferably between about 1.0 and 1.5 mm thick ×1.1 and 1.5 cm. wide ×9.0cm and 9.5 cm. long.

The transenamel bleaching tape systems as described above were exposed to plurality of 12 numbered sealed beakers comprising water having the temperature of about 50° C. At five minutes the sample in beaker #1 was removed and a cross-sectional specimen viewed under the magnification of 100× and 400× on a Nicon Labophot POL compound color microscope. The osmotic pressure of each sample was calculated using the Van't Hoff equation cited below. At five minutes intervals thereafter samples #2-#12, were removed and all 12 beakers were viewed under the microscope. During the bleaching process approximately 85% of the bleaching actives were activated by the alkaline elements in the first 10-15 minutes as measured by the indicator color change from yellow (acid) to pink (base). The latent bleaching actives were much slower to react then are the alkaline elements which instantly upon dissolution degrade the bleaching actives to generate small bubbles in the beakers. During the initial 2 to 5 minutes of water exposure the bleaching actives reacting with the alkaline elements is minimal. After the first five minutes the alkaline elements react with the bleaching actives and the bleaching curve begins to rise and then starts to level off at about 35 minutes due to the complete neutralization of the hydrophilic layer by the water permeable alkaline sublayer.

The osmotic pressure within the swelling alkaline sublayer of the laminate was calculated by using modified Van't Hoff equation:

$$P = \frac{c ( volume ) \times R \times T}{Mn} ( bar )$$

where

P=osmotic pressure

c=volume in cc/ml H20+PPVP

R=gas constant 0.0821 bar/degree

T=absolute temperature, 0K

Mn=number average of the molecular weight

The following are the osmotic pressure results for the 12 samples taken of the swelled alkaline sublayer containing 10% Crospovidone M:

| | Volume | Osmotic pressure | Osmotic pressure |
|---|---|---|---|
| Sample | water + PPVP | (calculated) | (measured) |
| 1 | 10 cc | ca. 0.05 bar | — |
| 2 | 10 cc | ca. 0.05 bar | — |
| 3 | 32 cc | ca. 1.55 bar | 1.60 bar |
| 4 | 35 cc | ca. 1.75 bar | 1.80 bar |
| 5 | 36 cc | ca. 1.80 bar | 1.85 bar |
| 6 | 37 cc | ca. 1.82 bar | 1.88 bar |
| 7 | 36 cc | ca. 1.85 bar | 1.90 bar |
| 8 | 38 cc | ca. 1.90 bar | 1.95 bar |
| 9 | 40 cc | ca. 1.95 bar | 2.00 bar |
| 10 | 40 cc | ca. 1.96 bar | 2.02 bar |
| 11 | 40 cc | ca. 2.00 bar | 2.06 bar |
| 12 | 40 cc | ca. 2.00 bar | 2.06 bar |

The above results illustrate the relationship between the increase of osmotic pressure and the decrease of pH in the alkaline sublayer. The chemical reactions of the above bleaching process can be appreciated more easily by referencing the changes in the pH in the hydrophilic layer during the bleaching stage under the conditions of the foregoing example. It becomes apparent that an important feature of the invention is sudden rise of osmotic pressure and gradual drop of pH in the alkaline layer and the rise of pH in the hydrophilic layer to pH about 7.5 due to the reverse osmosis of the alkaline elements into the hydrophilic bleaching layer. As water contacts the alkaline material in the permeable sublayer the pH approaches its highest value. About 85% of the alkaline elements are dissolvedand activated in about 15 minutes which corresponds to the greatest increase in the osmotic pressure. However, as alkaline elements are dissolved the pH value of the hydrophilic layer increase to about between 7.5 and 8.5 to affect the bleaching actives. With further gradual increase of pH from 7.5 to 8.5 the activity of the bleaching actives is increased so that the greatest amount of free-oxygen generation of the inactive, inert, latent bleaching actives occurs approximately during the 15 to 55 minutes of the process.

Each of the above 12 test cross-sectional specimens were examined under the magnification of 100× and 400× on a Nicon Labophot-POL compound microscope to determine what effect the osmotic pressure within the hydrophobic but moisture and saliva permeable swelling alkaline sublayer will have upon the relative spacial relationship of the alkaline elements. The results somewhat parallel the outcome of the above osmotic pressure. The beaker #1 (5 minutes of water exposure) manifested alkaline crystals of the prismatic form and characterized as somewhat with closed interatomic distances. Their space lattices are chiefly uniform, regular in shape and nonporous which makes the alkaline crystals harder and stronger but less water soluble. There is no evidence of water “islands” in the lattice matrix. The bleaching layer manifested a thick yellow floculant precipitate of the mixed hydrogen peroxide and soluble polyvinylpyrrolidone. The specimen in beaker #3 (15 minutes of water exposure) under the microscope the nuclei of crystallization manifested unproportional mixture of prismatic and monoclinic space lattice rather than solely prismatic. The nuclei are of somewhat wider interatomic distances with multiple water “islands” in the lattice matrix. This spacing of nuclei of crystallization and the presence of water in the matrix increases the osmotic pressure within the sublayer and prevents the growth of the needle-like crystals from entangling with each other. This effect contributes to to a more soluble and hygroscopic alkaline elements to diffuse into the bleaching layer to activate the bleaching elements. The bleaching layer when viewed under the microscope illustrates a more viscous, rather than floculant precipitate, with significant oxygen bubbles of various sizes throughout the water “islands”. From specimens in beakers #4 to #12 viewed under the microscope manifest increase of water “islands” with increasing numbers of oxygen bubbles throughout the “islands” and interatomic distances in the lattices much farther apart from each other which contributes to the high water solubility of both the alkaline and bleaching elements. At this stage the floculant precipitate in the bleaching layer is completely replaced with a homogeneous blend of yellowish-pink viscous composition indicating acid-base reaction and hydroxyl ion—hydrogen ion exchange. The bleaching layer is completely occupied with great number of oxygen bubbles of differing sizes. It appears as if the two layers, the alkaline and bleaching layers, have become one.

Another alternative method of preparing the transenamel bleaching system of the present invention may be accomplished as follows:

Method II

1. 0.5 mm thick, 37.5 g. of film forming acrylic polymer (Avalure AC/B. F. Goodrich Co.) was dissolved in 100 g. of hydroalcoholic solution (comprising 60 g. ethanol and 40 g. purified water) with gentle stirring. 10% of NaOH freshly prepared solution was added until a pH of 7.5 was attained. When completely in solution the viscosity of the composition is between about 500,000 and 1,500,000 cps. Approximately 0.5 grams/cm of the copolymer was casted on glass surface to a thin film approximately 0.50 mm thick. The film was then allowed to semi-harden to a tacky consistency. 2. In a separate glass beaker 50 g. of film forming PVP/Polycarbamyl Polyglycol Ester (Pecogel H-12/Phoenix Chemical, Inc.) and 5 grams of poly(ethylene oxide) were dissolved in 100 ml purified water that had the pH adjusted to about 6.5 with 4 g of sodium bicarbonate and stirred until the polymer was completely dissolved. To this mixture 44 grams of carbamide peroxide were added and gently stirred to insure homogeneous dispersion throughout The active film forming dispersion has the viscosity of about 500,000 and 1,500,000 cps. 3. The active solution 2 was casted upon the still tacky polymer in step 1 at a rate of 0.5 grams/cm₂ to produce a film 0.75 mm thick and forms a thin layer of inclusion complex. The total thickness of the transenamel bleaching system prior to drying is about 1.25 mm. After drying and evaporation of the solvents, water and alcohol, the thickness of the film generally is between 0.50 and 1.0 mm.

Examples of hydrophobic, water and saliva impermeable film laminate are compounds such as polyethylene, polypropylene, polystyrene, polyurethane, and similar materials.

Examples of the preferred materials for use as the hydrophobic and slightly moisture permeable continuous phase of the copolymer of the present invention are pharmacologically innocuous, bleach-stable organic substances, polymers and copolymers selected from a group consisting of polymerizing acrylonitrile; butadiene and styrene monomers; acetal copolymers; acetal homopolymers; acrylics; allyls; aminos; cellulosics; fluoroplastics; furans; ionomers; nitrile barrier resins; nylons; phenolic; phenylene oxide based resins; poly(amide-imide); polyaryl ethers; polyaryl sulfones; polybutadienes; polybutylenes; polycarbonates; polyesters; polyethersulfones; polyimides; polyphenylene sulfides; polysulfones; silicones; salts of heavy metals cellulose sulfates; paper and similar compounds and mixtures thereof.

The most suitable members of the above classes to make the water insoluble continuous hydrophobic phase of the present invention comprise poly(acrylic acid), poly(vinyl acetate), and nonionic hydrogels such as VP/dimethylaminoethylmethacrylate Polycarbamyl polyglycol ester, VP/dimethiconylacrylate polycarbamylpolyglycol ester, polymethacrylic acid esters, poly(ethylene oxide), insoluble polyvinylpyrrolidone and mixtures thereof.

Examples of the preferred material for use as the water soluble phase in the complexion of the copolymer of the present invention wherein the active ingredients, or drugs, are deposited either by dispersion, emulsion, distribution or dissolution are selected from a group of compounds that are hydrophilic, pharmacologically innocuous, bleach-stable, pseudoplastic organic and inorganic compounds and that they may be formulated to be readily soluble in water or saliva or have sustained release of the active ingredients over a long period of time, materials that are polymers or copolymers such as polyvinylpyrolidone; poly(-vinyl methylether/maleic anhydrite) copolymer half amide; polyethylene oxide; polyvinyl alcohol; hydrolyzed cellulose esters such as cellulose acetate hydrolyzed to an acetyl content of 19 to 26 percent; hydroxyethyl cellulose, hydroxypropyl cellulose, methyl cellulose, sodium carboxymethyl cellulose, etc.; polyacrylamide or an imidized polyarylamide; tributyl phthalate; substituted acrylates that are soluble in alkaline water such as carbopol: ethylene vinyl acetate copolymer; methacrylic acid copolymer; block polymers and copolymers and mixtures thereof

Examples of the most suitable members for use as the hydrophilic phase and as adhesive agents to affix the transenamel system of the present invention to the enamel surfaces are bleach-stable polymers or copolymers such as polyvinylpyrrolidone, propylene glycol alginate, substituted cellulose, block copolymers, polyvinyl alcohol, polyethylene oxide, blend of xanthan gum/glucose/carrageenan, low molecular weight PVP/polycarbamyl polyglycol ester, and mixtures thereof.

Examples of the bleaching agents suitable for use in the present transenamel system of the present invention comprise one or more of those peroxygens that are physiologically and pharmacologically tolerable with reference to a particular oral utility of the system of the present invention and preferably should be water soluble and miscible. It should be solid and be dispersed, emulated, suspended or otherwise distributed in the hydrophilic matrix. It has been found in connection with the present invention that the most suitable bleaching substances are peroxygens such as hardened or crystallized hydrogen peroxide, carbamide peroxide (urea peroxide), calcium peroxide,sodium peroxide, sodium carbonate peroxide, stabilized chlorine dioxide, stabilized chloride dioxide, sodium perborate monohydrate, sodium percarbonate, peroxyhydrate magnesium peroxide; peracids such as magnesium monoperoxyphthalate, diperoxydodecanedioic acid, peroxydiphosphate, etc; enzymes such as oxydoreductaze, lysozyme, etc. and similar inorganic and organic compounds which yield oxygen when exposed to air and water and saliva. The concentration of the bleaching agent may vary depending upon the reactivity. The most effective amount may be present in an operative range from about 1% and 75% with respect to the total weight of the matrix composition

The most suitable bleaching and whitening agent in accordance with the improved present invention are dried hydrogen peroxide and carbamine peroxide. Peroxygens such as hydrogen peroxide, in liquid or gel state are not useful in the present invention due to their instability and loss of potency via evaporation in compositions stored over an extended period of time. It is estimated that hydrogen peroxide looses approximately 1 percent to 2 percent of its potency every month in storage.

Examples of some fluoride yielding compounds useful in the transenamel system of the present invention are sodium fluoride, calcium fluoride, sodium monofluorophosphate, stannous fluoride, hydrogen fluoride, etc. and mixtures thereof and the most effective amount may be present in an operative range from about 0.01% and about 25% with respect to the total weight of the matrix composition.

In addition, in accordance with the present invention components specified herein may have additives performing specific desired functions such as alkaline catalysts that are nontoxic when used in the suggested amounts, the said alkaline catalysts comprise a member of a group consisting substances that have a pH range of between about 6.5 and 14 such as sodium hydroxide, potassium hydroxide, calcium hydroxide, ammonium hydroxide, triethanolamine, trisodium phosphate, sodium carbonate, calcium carbonate, sodium bicarbonate, sodium stannate, etc present in an operative range sufficient to adjust the pH of the dissolving water solution between about 6.5 and 10.5; sweetening agents of about 0.05% and 1%; preservatives, 0.05% and 2%; flavors, 0.05% and 5%; plasticizers, 0.1% and 10%; alcohols, 5% and 80%; surface active agents 0.25% and 25% and other formulating agents.

### EXAMPLES

In order to point out more fully the nature of the present invention, the following specific examples are given as an illustrative embodiment of the present drug delivery systems produced thereby.

### Example 1

A two phase, clear, transparent, transenamel bleaching system (tape) of the present invention was prepared according to the following steps:

A. Hydrophobic, Impermeable Laminate (Impermeable Protective Layer)

| | Weight % |
|---|---|
| Polyethylene | 45.00 |
| Acetone | 55.00 |

Upon dissolution of the polyethylene in Acetone the resultant viscous composition was casted on glass surface at a rate of 0.5 grams/cm2 to provide a thickness of about 0.5 mm upon drying.

B. Saliva and Moisture Permeable Swelling Alkaline Laminate Sublayer (Permeable Alkaline Layer)

| | | Weight % |
|---|---|---|
| Crospovidone M* | | 10.00 |
| Water, pure | | 78.85 |
| Sodium saccharine | | 0.40 |
| Flavor | | 1.25 |
| Ammonium hydroxide | q.s. | 9.50 |

*Film forming insoluble, swelling polyvinylpyrrolidone polymer. BASF.

Crospovidone M was dissolved in warm water that has been neutralized to a pH between 9 and 10 with ammonium hydroxide with gentle stirring to prevent foaming. Sodium saccharine and flavor were added into a small amount of water (10 ml) to dissolve and then mixed with the aqueous solution to form a viscous composition. The viscosity of the composition was measured to be 150,000 csp. Ammonium hydroxide being volatile compound will completely evaporate during drying to provide a permanent water insoluble but moisture and saliva permeable film with swelling properties.

The composition was casted on still wet coating laminate described in A above at a rate of 0.5 grams/cm to provide a thickness of about 0.5 mm. It was allowed to dry to a tacky consistency before coated by the dispersion layer C below.

C. Hydrophilic Dispersion Layer (Soluble Bleaching Layer)

| | | Weight |
|---|---|---|
| Polyvinylpyrrolidone (PVP K-90)* | | 10.00 |
| Lipomoist-2013** | | 10.00 |
| Water, purified | | 52.68 |
| Carbamide peroxide | | 25.00 |
| Sodium saccharine | | 0.20 |
| Sodium benzoate | | 0.40 |
| Benzoic acid | | 0.20 |
| Sodium lauryl sulfate | | 0.52 |
| Flavor | | 1.00 |
| Sodium hydroxide (10%) | q.s. | pH 6.5 |

*Water soluble film forming grade of PVP provided by ISP, Inc.
**Film forming moisturizing molecular submicrodispersion of vegetal polymers provided by Centerchem, Inc.

Polyvinylpyrrolidone and Lipomoist-2013 were slowly added with stirring into a container containing purified water until a viscous solution is obtained. Sodium saccharine, sodium benzoate, benzoic acid, sodium lauryl sulfate and flavor were then added and dissolved before carbamide peroxide was added slowly with slow mixing until all of peroxide is homogeneously distributed. When completely mixed the water soluble phase was coated immediately on the still tacky composition layer of B above at a rate of 1.0 gram/cm2 to produce a thickness of 1.0 mm which includes the inclusion complex formed at the junction of the two phases. The completed composition was then dried under low heat until non-tacky to touch. The total thickness of the dried tape is 1.5 mm. The transenamel system was then cut into individual tapes having dimensions of approximately 1.2 cm wide and 8.5 cm long before packaging into moisture impermeable bleach-stable pouch of required size prior to being used.

The carbamide peroxide of this example provides concentration of 16.66 percent with respect to the total weight of the composition or 6.0% in hydrogen peroxide equivalency.

### Example 2

A clear, two phase, transparent, flexible, transenamel bleaching system was prepared in the following manner:

A. Hydrophobic, Impermeable Laminate Layer (Impermeable Protective Layer)

| | Weight % |
|---|---|
| Polystyrene | 55.00 |
| Acetone | 45.00 |

Cast the resultant viscous solution in the same manner as in Example 1.

B. Saliva and Moisture Permeable Swelling Laminate Alkaline Sublayer. (Permeable Alkaline Layer)

| | | Weight % |
|---|---|---|
| Sentry ™ Plus Chewing Gum Base GB-40* | | 35.00 |
| Alcohol | | 63.55 |
| Sodium saccharine | | 0.20 |
| Flavor | | 1.25 |
| Sodium hydroxide (10%) | q.s. | pH 8.50 |

*Polyvinyl acetate obtained from Union Carbide Corporation
The composition was prepared in the same manner as in Example 1 with same dimensions.

C. Hydrophilic Dispersion Layer (Soluble Bleaching Layer)

| | % Weight |
|---|---|
| Polyvinylpyrrolidone (Collide −30)* | 25.00 |
| Isopropanol | 30.00 |
| Water | 10.92 |
| Propylene glycol | 6.50 |
| Hydrogen peroxide (50%) | 5.00 |
| Carbamide peroxide | 15.00 |
| Polyethylene oxide | 4.00 |
| Carbopol | 1.50 |
| Sodium saccharine | 0.18 |
| Sodium benzoate | 0.40 |
| Chewing gum flavor | 1.50 |

*Kollidon - 30 provided by BASF Corporation.

The finished product of Example 2 was similar to that of Example 1 with like dimensions, however, the ratio of B to A was 1:1. It provided 2.5% hydrogen peroxide and 7.5% carbamide peroxide.

### Example 3

Example 1 was repeated except polyvinyl acetate polymer in the hydrophobic, moisture permeable laminate (sublayer) was replaced with PVP/dimethiconylacrylate/polycarbanyl polyglycol ester and Lipomoist-2013 (™) in the water soluble discontinuous dispersion (active phase) was replaced with hydroxyethyl cellulose. Tape samples of this example having measurements 1.00 mm thick ×1.2 cm wide and 9.0 cm long were used on trial subjects. The subjects were asked to apply the side of the tape coated with the active phase which comprises the adhesive substances when wetted to the surfaces, preferably moistened with saliva, of the teeth and thus retain the tapes on the surface of the upper anteriors for 45 minutes and then removed. The polyvinylpyrrolidone (PVP) in the water insoluble substrate dissolved quicker than vinyl acetate (Example 1) and acrylic polymer (Example 2) which allowed some of the peroxide to react with mucous membranes in contact to produce minor chemical irritations.

### Example 4

A set of transenamel bleaching tapes were made in which the water insoluble phase contained the same ingredients and in the same amounts as in Example 1(A) and in which the bleaching active carbamide peroxide in the amount of 15 percent was suspended in a plurality of compositions and dried in accordance of the present invention. The study was to determine the film forming characteristics of each ingredient for use in the present improved invention and the time (in minutes) for dissolution of each. All dried samples were of equal dimensions: 1.5 mm thick ×5 cm square and placed in separate beaker containing 50 ml purified water.

TABLE I

| Sample | Compositions | Film Former | Time(in minutes) |
|---|---|---|---|
| A. | Polyvinylpyrrolidone | yes | 70 |
| B. | Carbopol | Slight | 45 |
| C. | Hydroxyethyl cellulose | yes | 18 |
| D. | Lipomoist-2013 | yes | 35 |
| E. | Polyvinyl acetate | yes | Insoluble |
| F. | Acrylic polymer | yes | Insoluble |
| G. | Locus bean gum | no | 10 |
| H. | Polyethylene oxide | yes | 5 |
| I. | Lipomoist-2013 and | yes | 28 |
| | Locus bean gum | | |
| J. | Hydroxypropyl cellulose | no | — |
| K. | Carbopol and Hydroxyethyl | no | — |
| | cellulose | | |
| L. | Guar gum | no | — |
| M. | Polyvinylpyrrolidone and | yes | 55 |
| | Lipomoist | | |

The above table indicates that compositions A, D, E, F, H and I being film formers and gelling agents when exposed to water, or saliva, are suitable as dispersion matrix for the actives of the present invention. However, compositions G, H and I dissolve too quickly to be provide sustained action of the bleaching agent carbamide peroxide for longer than 30 minutes, therefore compositions A and M are the most suitable as film forming and gel forming compounds. Formation of gel is important in the concept of the present invention in that it provides an environment for the crystalline carbamide peroxide to be dissolved quicker and provide fresh composition of the bleaching agent.

All of the presently commercially available tooth whitening compositions, either in dentifrice formulations or in the case of WhiteStrips, are without exception in gel form prior to their utilization is the oral cavity or during the bleaching process. The present invention provides a system wherein the bleaching actives are in a film of flexural stiffness and only a portion of the film becomes semi-gel when applied topically to selected tooth surfaces.

In utilizing the concept of the present invention wherein the method applies a covariance structure that accounts for the heterogeneity of solubility variance and correlation of dissolution across time points a film constructed in Example 1 is topically applied to a selected tooth surfaces or to plurality of adjacent teeth. The film comprises water insoluble continuous phase (substrate) and equivalent concentration of the bleaching actives throughout the structure of the discontinuous dispersion phase which is on the side of the tape adjacent to the enamel surfaces to provide for a quick onset of bleaching action.

### Example 5

Transenamel bleaching tapes in accordance with the present invention were prepared in the following manner:

A. Hydrophobic, Impermeable Laminate Layer

Commercially available polyethylene film.

B. Saliva and Moisture Permeable Swelling Alkaline Laminate Sublayer

| | | Weight % (g) |
|---|---|---|
| Polyvinyl acetate | | 45.00 |
| Water | | 53.35 |
| Sodium saccharine | | 0.40 |
| Flavor | | 1.25 |
| Sodium hydroxide (10%)* | q.s. | pH 7.5 |

*In this Example sodium hydroxide was used to neutralize the composition rather than ammonium hydroxide, as in Example 1. Dried form neutralized with sodium hydroxide will provide a temporary coating that is readily permeable to air and moisture and is soluble in alkaline solutions.

The partially dried sublayer was then coated with the following.

C. Hydrophilic Dispersion Layer (.Bleaching Layer)

containing the following ingredients:

| | | Weight % (g) |
|---|---|---|
| Polyvinylpyrrolidone | | 10.00 |
| Lipomoist-2013 | | 10.00 |
| Water, purified | | 52.68 |
| Carbamide peroxide | | 25.00 |
| Sodium saccharine | | 0.20 |
| Sodium benzoate | | 0.40 |
| Bezoic acid | | 0.20 |
| Sodium lauryl sulfate | | 0.52 |
| Flavor | | 1.00 |
| Sodium hydroxide (10%) | q.s. | pH 6.5 |

The hydrophilic bleaching layer (C) was overlaid by

C. Release Liner.

Non-adhesive acrylic film that must be removed prior to use.

The entire bleaching system then may be covered by a bleach-stable wrapper that protects the system from evaporation of the ingredients and it must be removed prior to use.

The concentration of the bleaching agent carbamide peroxide in the hydrophilic copolymer matrix emulsion composition was 25%. The bleaching composition of the matrix emulsion was coated on the hydrophobic moisture permeable sublayer (B) above at a rate of 5 cc per 6 mm ×1.4 mm area. The system was dried at heat of not more than 100° F. until dry. It then became more fluid when exposed to the saliva in the oral cavity and it became pliable enough to allow the flow of the newly formed carbamide peroxide gel under pressure into the interproximal spaces in the oral cavity. The total thickness of the system is less than 2 mm.

The rate of delivery of the bleaching agent, carbamide peroxide now degraded to hydrogen peroxide, to the patient from each system (31 mcg/cm2/hr) is proportional to the bleaching-gel matrix and to the concentration of the peroxygen. Less than about 50% of the total amount of the hydrogen peroxide and about 75% of the hydrophilic matrix remain in the system every 20 minutes after application. Approximately 25% of the hydrophilic matrix and 15% of the hydrogen peroxide volatile from the edge of the system every 20 minutes

The amount of the hydrogen peroxide eliminated by evaporation from the edges of the system was determined by titrating the matrix with indigo-carmine indicator after every 20 minutes of usage and then subtracting the resultant number from the original total baseline concentration of the peroxide.

To determine the active oxygen content of the peroxygen, the usual method for hydrogen peroxide was used; i.e. the titration with potassium permanganate in a sulfuric acid solution. A 1 ml of potassium permanganate solution [c(KMnO4)=0.02 mole/litter] is equivalent to 0.8 mg of active oxygen, 1.7 mg. of hydrogen peroxide or 4.7 mg of carbamide peroxide. Every 20 minutes of usage in the oral cavity the tape was removed and examined for the remained peroxide and the resultant number was subtracted from the original total baseline. A new transenamel tape was used for the second 20 minutes less the amount of peroxide used-up. The following table reflects the results of that study.

TABLE II

| | Dose Absorbed | System Area | Total Peroxide |
|---|---|---|---|
| Treatment Dose | in 1 hr (g/hr) | (6 mm × 1.4 mm) | Content (g) |
| First 20 minutes | 0.442 | 8.4 | 1.0 |
| Second 20 minutes | 0.243 | 6.3 | 0.55 |
| Third 20 minutes | 0.136 | 4.7 | 0.31 |

The amphiphilic hydrogen peroxide, the chief bleaching agent in this example, has lypophilic (oxygen) and hydrophilic (water) molecules in its formula which the lypophilic molecule binds stereoselectively to lipid composition of the pellicle (smear layer) of the enamel initially and by the process of lypolysis dissolves the pellicle thereby allowing the generated oxygen to attack and cleaves all double-bond stains to form lighter shades of the intermediates. The oxygen seeks and destroys any double-bond in the enamel rods and dentinal tubules to oxidize them and the process of bleaching progresses until there are no longer double-bonds or until oxygen is used-up in reaction with hydroxyapatite to form white 2-calcium oxyapatite. This chemical reaction is reversible. The 2-calcium oxyapatite, the intermediate of the bleaching process, will remain white until the life-time ongoing remineralization and accretion process of calcium and phosphorus deposition on the skeletal tissues, including teeth, will replace gradually some of the oxygen in the 2-calcium oxyapatite to reform the inorganic calcium hydroxyapatite. Therefore, the bleaching of teeth process should be repeated periodically, usually every 2-5 months.

### Example 6

A clear, transparent control-release transenamel bleach delivery system was prepared containing the following multilayers:

1. Hydrophobic Layer (Substrate) of Hydrophobic Poly(Vinyl Chloride) About 1.5 mil Thick.

2. Hydrophilic Layer (Bleaching Layer)

| | Weight % (g) |
|---|---|
| Purified water | 56.30 |
| Lipomoist-2013 | 11.50 |
| Carbamide peroxide | 30.00 |
| Polyvinylpyrrolidone | 0.50 |
| Triethanolamine | 0.30 |
| Zinc chloride | 1.00 |
| Sodium saccharine | 0.20 |
| Methyl and propyl paraben | 0.20 |

In this example, zinc chloride (to reduce gingival irritation), sodium saccharine (sweetener), methyl and propyl paraben (preservatives) are first dissolved in the purified water before thickeners Lipomoist-2013 and polyvinylpyrrolidone are dissolved with gentle stirring to reduce air bubbles formation. Triethanolamine was then added to raise the pH of the solution to about 7.5 and thicken the composition. Carbamide peroxide was added slowly with constant stirring to avoid formation of islands of peroxide.

10 cc of the very viscous composition was coated on a rectangular 6 mm×2.8 mm film of the poly(vinyl chloride) occlusive backing layer.

3. Protective Release Liner.

A disposable non-adhesive, hydrophobic acrylate liner overlays the Contact Adhesive Layer and must be removed prior to use.

The concentration of carbamide peroxide in this example was 12%.

Carbamide peroxide is the active ingredient; other components of the system are pharmacologically inactive.

Transenamel delivery system of this example is topically applied on the enamel surfaces of the eight upper (maxillary) anterior teeth (left and right centrals, laterals cuspids and first bicuspids) starting at the anterior gingival margins and gradually extending downward with finger pressure to assure complete coverage of the teeth, including interproximally, with the pseodoplastic matrix composition and then, when the incisal part of the teeth is reached, reversing the direction of application by extending the transenamel system upward up to the lingual gingival margin.

The rate of delivery of peroxide to the enamel surfaces from each system is about 40 microg/cm2-h. Approximately 85% of the total amount of peroxide remains in the system 8 hours after application. The dose of peroxide absorbed from the system represents 24% of the amount released in 8 hours. The other 76% volatilizes from the edge of the system.

TABLE II

| Dose Absorbed in 1 hour | System Area | Total Peroxide Content |
|---|---|---|
| (g/1 hr) | (mm) | (g) |
| After 20 min. 0.360 | 16.8 | 1.0 |
| After 40 min. 0.347 | 12.3 | 0.964 |
| After 60 min. 0.334 | 9.2 | 0.929 |

Pharmacological action of carbamide peroxide and related peroxygens is limited to binding stereoselectively and temporarily to hydroxyapatite crystals of enamel and cleaving double-bonded stains. Its action is transient for a limited time and its chemistry is reversible. When oxygen is completely used-up, normally within two to four months, the hydroxyapatite crystals are continually being remineralized by calcium and phosphorus deposition via the blood plasma and the color of the enamel reverts to its natural shade or hue.

There is no evidence of any pharmacodynamic action of hydrogen peroxide when applied typically to the enamel surface. Its cosmetic action is limited only to the hydrohyapatite crystals of the enamel and there is no absorption of hydrogen peroxide in the blood plasma.

Pharmacokinetically the major eliminating organ of peroxygens when topically applied to the enamel surfaces is the skeletal, including teeth. The process of elimination is by evaporation of oxygen in the atmosphere. There is no significant metabolism of peroxygen in the enamel; the only possibility of alteration of plasma protein is when peroxygens penetrate into the pulp chamber to affect inflammation and possibly hyperthrophy and even then it would not be expected to have significant consequences.

### Example 7

A transenamel delivery system was made consisting the following:

A. Hydrophobic, Impermeable Laminate Layer.

Laminated polyester water and saliva hydrophobic film.

B. Saliva and Moisture Permeable Swelling Laminate Alkaline Sublayer.

| | Weight % |
|---|---|
| Water | 65.00 |
| Acrylic Acid | 10.00 |
| Ammonium Hydroxide | 3.50 |
| Polyethylene Oxide | 21.50 |

C. Rate Control Mambrane

| | Weight % |
|---|---|
| Polyvinyl Acetate | 15.00 |
| Water | 65.00 |
| Alcohol | 20.00 |

C. Hydrophilic Phase (Bleaching Phase)

consisting:

| | Weight % (g) |
|---|---|
| Nonwoven absorbable material | 15.0 |
| Poly(ethylene oxide) | 12.5 |
| Water | 59.5 |
| Sodium monofluorophosphate | 5.0 |
| Polyvinylpyrolidone | 8.0 |

B. Protective Release Liner

Non-adhesive laminate paper overlies the matrix and must be removed prior to applying to tooth enamel.

### Example 8

Example 1 was repeated except that the water soluble phase contained the following ingredients:

| | Weight % (g) |
|---|---|
| Hydrogen peroxide (50%) | 10.00 |
| Carbamide peroxide | 20.00 |
| Pluronic | 5.50 |
| Water, deionized | 55.00 |
| Glycerin | 2.50 |
| Sodium saccharine | 0.20 |
| Methyl and propyl paraben | 1.30 |
| Flavor | 1.50 |
| Polyvinylpyrrolidone | 4.00 |

The concentration of the bleaching agent, hydrogen peroxide, in this example was 5% and carbamide peroxide 7%.

### Example 9

A double blind study of twenty individuals was conducted to evaluate the efficacy and safety of a transenamel drug delivery system containing a bleaching agent and an alkaline activator (catalyst) dispersed in the substrate film when used topically on selected enamel tooth surfaces and compare it to a gel composition of similar concentration. Two models were used: (a) a polymer or copolymer gel coated with an active phase containing hydrogen peroxide (10%) and (b) trial composition comprising a test polymer or copolymer film coated with an active phase containing hydrogen peroxide (10%). The color change and gingival indices were used to determine changes, and thereby the safety and efficacy of the test model. Examinations for both indices were made twice the first week to determine the pre-experimental gingival and tooth-shade status of each participant in Phase I (pre-experimental) For Phase II (experimental). The subjects were then divided into two groups: Group I used bleaching system made in accordance with the present invention comprising hydrogen peroxide in the active phase coated on a substrate having the pH of 8.5 and Group II used alkalinized substrate having pH of 8.5 coated with hydrogen peroxide gel. Gingival margins (for safety) and enamel color change (for efficacy) indices were scored for each group three times a week for two weeks and compared to the indices obtained in the pre-experimental trial to determine the safety and efficacy of each model.

All participants presented the following characteristics:

Inclusion Criteria:

1. Normal oral mucosa/minimal gingivitis.

2. In good general health.

3. Must have at least sixteen natural teeth.

4. Had prophylaxis one week prior to the study.

5. Were not undergoing extensive restorative treatment during the study.

6. Able to sign informed consent and willing to adhere to study protocol.

Exclusion Criteria:

1. By examination, free of significant signs of irritation, inflammation or other pathology.

2. Past history of severe reaction to bleaching products.

3. Systemic diseases (e.g. diabetes) which in the opinion of the Investigator, would warrant exclusion.

4. Pregnant and lactating women.

The condition of the marginal gingiva is assigned the applicable index ranging from zero through four:

0. Normal appearance of marginal gingiva of the treated teeth in color and structure. On palpation with a pocket probe, the gingiva is firm and does not bleed. Absence of inflammation.

1. Barely visible erythema.

2. Slight erythema plus slight edema.

3. Moderate erythema and/or edema.

4. Severe inflammation/irritation.

The bleaching of the selected teeth is scored on a zero to three scale using the Vita Shade Guide with Al being the lightest.

0. Corresponds to A2.

1. Corresponds to A3.

2. Corresponds to A4.

3. No improvement

Formulas:

| Control (Group I) | % | Experimental (Group II) | |
|---|---|---|---|
| Hydrophilic Phase | | Hydrophilic Phase | |
| Deionized water | 48.9 | Deionized water | 37.0 |
| Polyvinylpyrrolidone | 29.4 | Polyvinylpyrrolidone | 18.1 |
| Sodium Saccharine | 0.2 | Sodium saccharine | 0.2 |
| Polyethylene Glycol | 14.3 | Polyethylene glycol | 1.5 |
| Tween 20 | 1.7 | Tween 20 | 1.0 |
| Flavor | 1.0 | Flavor | 1.0 |
| Carbopol | 2.5 | Carbopol | 2.5 |
| Sodium Hydroxide | 2.00 | Hydrogen Peroxide | 27.0 |
| | | Sodium Hydroxide (10%) | 2.0 |

After analyzing the data statistically the code was broken. There was no significant improvement in either index at the initial appointment and at the end of the pre-trial examinations (Phase I). The improvement was significantly greater in the group using alkaline activator sodium hydroxide with hydrogen peroxide film (Group I) than that in Group II for both bleaching and gingival measures. Scores for Group I (hydrogen peroxide film) decreased significantly at the fourth examination (second week) and continued to improve at the eight examination (end of trial); whereas, the scores for the Group II (hydrogen peroxide and gel composition) showed slight improvement after the fourth examination. The bleaching superiority and lack of gingival irritation of the transenamel drug delivery system comprising hydrogen peroxide and sodium hydroxide for topical application on selected tooth surfaces was apparent at this time.

The trial of this example makes the following conclusions:

1. A transenamel drug delivery system comprising a dry peroxygen with an alkaline activator is more effective as a treatment model on the bleaching of teeth. The color indices of the participants using it were significantly improved than the indices of the participants using non-crystalline gel bleaching composition, at the third and sixth examination.

2. A transenamel drug delivery system comprising a dry peroxygen with an alkaline activator is safe on the gingival margins of the treated teeth. The gingival indices of the participants using it did not change significantly from the pre-trial phase.

In summary, the transenamel drug delivery systems of the present invention relates to a semi-rigid sustained release film unit of selected active agents and built-in hydroxyl ions—hydrogen ions exchange capability when used for topical application on selected tooth surfaces and which does not require refrigeration prior to use comprising (1) an effective amount of bleach-stable laminate material comprising (a) an effective amount of one saliva and moisture impermeable layer and (b) an effective amount of one saliva and moisture permeable swelling alkaline sublayer the sublayer having the pH adjusted to about 6.5 and 10.5 the laminate being present at the operative range from about 2.5 and 75 percent with respect to the total weight of the composition and (2) an effective amount of a hydrophilic acid layer consisting two or more bleach-stable hydrophilic substances having different solubilities and wherein the pH is adjusted between 4.5 and 6.8 and being present at the operative range from about 2.5 and 75 percent with respect to the total weight of the composition said and dispersed, emulsed, dissolved or otherwise distributed throughout said discontinuous phase an effective amount of one or more peroxygens present in the operative range from about 1.5% and 55% with respect to the total weight of the composition. The tapes are made by casting one of the phases, preferably the substrate, on non-stick surface, such as glass, and allow it to dry to a tacky state before coating at with the active phase at low heat air. The dried rigid and semi-rigid tapes then can be cut to any desired shapes and sizes suitable for the intended use. A hydrophobic release liner either of polymer or copolymer composition or paper covering the bleaching layer that must be removed prior to use may be an adjunct part of the transenamel system tapes. The entire system may be covered by a moisture barrier pouch (wrapper) of plastic material which also must be removed prior to use. The semi-rigid transenamel bleach delivery system when applied topically to selected tooth surfaces will adhere aggressively to the enamel and when exposed to the saliva and moisture in the oral cavity transforms into a soft and flexible to conform to the contour of the selected tooth surfaces. The hydrophilic bleach layer containing the latent crystalline bleaching elements are wetted and dissolved by oral saliva and upon exposure to the alkaline elements will increase the pH of the bleaching layer to about 6.5-8.5 thus degrading the latent bleaching elements to generate free oxygen that is readily available for uptake by the enamel rods to affect tooth whitening color change. The system of the present invention may provide sustained release of the active bleaching elements, only when wetted by oral saliva and exposed to the catalyst elements, for a prolonged period of time without adversely effecting the surrounding gingival margins and other oral soft tissues and can easily be removed by stripping it off the enamel surfaces.

In utilizing the transenamel bleaching system of the present invention and when exposed to saliva as described above the activated alkaline ions (hydroxyl group) in the alkaline layer migrate into the bleaching layer to instantly react chemically with the heretofore inactive bleaching actives to effect generation of free oxygen and affect whitening of enamel surfaces. The activation time of the bleaching actives is between about 5 and 10 minutes this is the time necessary for the saliva to enter the alkaline layer and start the disso I ution of the alkaline elements, This is shown graphically in FIG. 5 where the solid line indicates almost immediate beginning. of dissolution of the bleaching layer which dissolves steadily until almost completely dissolved in about 60 minutes. The broken line indicates saliva and moisture absorption of the alkaline layer. It starts at about between 5 and 10 minutes and gradually swells, with some dissolution, as it absorbs saliva and moisture and r eleases the activated alkaline elements to diffuse into the bleaching layer. It is the beginning of the osmosis stage and the bleaching stage of the invention illustrated by the shaded area. This stage of hydrogen ions hydroxyl ions exchange ends at about 60 minutes when most of the bleaching layer is dissolved. The bleaching stage starts simultaneously with the osmosis stage. The pH of the transenamel bleaching tapes before the activation stage is neutral. When the tape after exposure to saliva and moisture in the oral cavity, in addition to normal oral enzymes catalase and peroxygenase, the hydrophilic bleaching layer begins to dissolve -and form a gel-like composition the pH decreases from 7 to nearly 5.5 and 6.5. However, when alkaline layer begins to dissolve or swell equal amounts of the alkaline elements will be brought into the chemical acid-base reaction to maintain the pH of the system between 5.5 and 6.5 temporarily. As the alkaline elements continue to diffuse into the bleaching layer the pH increases sharply. The above proccessing can be appreciated more easily by reference to FIG. 6 wherein the shaded area illustrates the relative acid-base equilibrium of the present invention. It is the period of prebleaching phase where the bleaching layer begjns to dissolve and the pH drops to about 5.5 and 6.5. It is the period between about 5 and less than 15 minutes after exposure to saliva and moisture and is the stage of relative acid-base equilibrium before the alkaline layer begins to activate the hydroxyl ions. Between 15 and 25 minutes there is steady increase in the pH value to about 9 and remains steady for the duration of the bleaching process.

The present invention may be embodied in other specific forms without departing from the spirit and essential characteristics. The described embodiments are to be considered in all respects only as illustrative and not restrictive. The scope of the invention is indicated by the appended claims rather than by the foregoing description. All changes which come within the meaning and range of equivalency of the claims are to be embraced within this scope. In view of the above disclosure, it will be noted that the several objectives of the invention are achieved and other advantageous results obtained, therefore, what I desire to claim and secure by Letters Patens is:

What is claimed is:

1. A sustained release transenamel bleach delivery oral composition having built-in hydroxyl ions—hydrogen ions exchange capability for topical application on teeth in an oral cavity comprising:
(a) an effective amount of bleach-stable carrier of active substances forming laminate layer comprising
(i) an effective amount of an outer saliva and moisture impermeable layer being present in the operative range of about 2.5% and 75% with respect to the total weight of the composition; and
(ii) an effective amount of an inner saliva and moisture permeable swelling alkaline sublayer of bleach-stable polymers or copolymers acting as latent hydroxyl ions donors and having the pH adjusted to between 6.5 and 10.5 being present in the operative range of about 2.5% to 75% with respect to the total weight of the composition; and
(b) an effective amount of bleach-stable hydrophilic acid forming layer acting as a hydroxyl ions receptor having the pH between about 4.5 and 6.8 and comprising organic and inorganic substances present in the operative range of about 2.5% and 75% with respect to the total weight of the composition wherein dispersed, emulsed, incorporated or otherwise distributed throughout said layer is an effective amount of one or more peroxygen compound present in the operative range of about 1.5% and 55% with respect to the total weight of the composition,
said transenamel bleaching system when topically applied to teeth and exposed to the saliva and moisture will effectively adhere to the teeth surfaces and wherein said saliva and moisture from the oral cavity will diffuse throughout said hydrophilic layer into said moisture permeable sublayer to activate said alkaline elements therein which via reverse osmosis, are forced into said bleach-stable hydrophilic acid forming layer comprising said peroxygen compound to raise the pH of said bleach-stable hydrophilic acid forming layer from about between 4.5 and 6.8 to about between 7.5 and 8.5 and activate the peroxygen compound to generate free oxygen for quick and effective bleaching.

2. A sustained release bleach delivery system of claim 1 comprising of one or more film forming laminate having layers which include said alkaline sublayer, said bealch stable hydrophilic acid forming layer and a removable protective layer.

3. A sustained release bleach delivery oral composition system of claim 1; wherein the forming permeable laminate alkaline sublayer has swelling properties which increase the osmotic pressure within said sublayer to force the polymers or copolymers by the process of diffusion into the hydrophilic bleach stable hydrophilic acid forming layer to activate the peroxygen compound.

4. A sustained release transenamel bleach delivery oral system of claim 3, wherein said peroxygen compound in the bleach stable hydrophilic acid forming layer is activated only when wetted by saliva and exposed to hydroxyl ions to convert said peroxygen compound into ionized active oxygen.

5. The composition of claim 1 wherein said acid bleach stable hydrophilic acid forming layer comprises at least one peroxygen compound present in said composition in an amount of between about 2 and 55 percent by weight based on the total weight of said composition.

6. The composition of claim 5 wherein said peroxygen compound is selected from the group consisting of hydrogen peroxide, carbamide peroxide, calcium peroxide, sodium peroxide, sodium carbonate peroxide, calcium carbonate peroxide, stabilized chloride peroxide, sodium perborate, sodium percarbonate, magnesium peroxide; peracids such as magnesium monoperoxyphthalate, diperoxydodecanedioic acid, peroxydiphosphate, sodium perborate monohydrate, sodium carbonate peroxyhydrate, etc; enzymes such as oxydoreductaze, lysozyme; and mixtures thereof.

7. The composition of claim 6 wherein said at least one of peroxygen compound is hydrogen peroxide and carbamide peroxide.

8. The composition of claim 1 wherein said peroxygen compound is hydrogen peroxide.

9. The composition of claim 1 wherein said at least one alkaline sublayer is selected from the group consisting of polymers or copolymers having pH between 7 and 14.

10. The composition of claim 9 wherein said polymers or copolymers comprise sodium hydroxide, ammonium hydroxide, potassium hydroxide, sodium bicarbonate, sodium phosphate dibasic, sodium phosphate tribasic, sodium carbonate and similar alkaline compounds.

11. The composition of claim 10 wherein said alkaline sublayer includes one of sodium hydroxide, ammonium hydroxide, sodium bicarbonate and sodium phosphate dibasic.

12. The composition in claim 11 wherein alkaline sublayer includes sodium hydroxide being present in said composition in an amount of about 0.5 to 45 percent by weight of said composition.

13. The composition of claim 2 wherein said removable protective layer is bleach-stable material and must be removed prior to use.

14. The composition of claim 1 further comprising one or more additional additives including fluoride ion releasing compounds, calcium ion releasing compounds, phosphorus ion releasing compounds, antimicrobials, sweeteners, flavors, colors, preservatives, alcohols, vitamins, minerals and adhesives.

15. The composition of claim 2 wherein said bleach-stable carrier of active substances forming laminate layer and said removable protective layer is water and saliva insoluble and water and saliva and moisture impermeable, bleach-stable, innocuous, and selected from the group consisting of polymerizing acrylonitrile; buyadiene and styrene monomers; acetal copolymers; acetal homopolymers; acrylics; allyls; aminos; cellulosics; fluoroplastics; furans; ionomers; nitrile barrier resins; nylons; phenolic; phenylene oxide based resins; poly(amide-imide); polyaryl ethers; polyaryl sulfones; polybutadienes; polybutalenes; polycarbonates; polyesters; polyethersurfones; polyethylenes; polyimides; polyphenylene sulfides; polypropylenes; polystyrenes; polysulfones; polyurethanes;polyvinyls; silicones; salts of heavy metal cellulose sulfates; laminated polyester; polyethylene/polyester/ethylene vinyl acetate copolymer; and aluminized paper.

16. A composition for bleaching tooth surfaces comprising:
(a) at least one saliva and moisture impermeable layer, and
(b) at least one saliva and moisture permeable alkaline layer, and
(c) a rate control membrane, and
(d) a hydrophilic bleaching layer.

17. A method for bleaching of tooth surface comprising a multilayered transenamel bleach delivery system having built-in hydroxyl ions—hydrogen ions exchange capability comprising (A) a laminate layer of one of polymers, copolymers and cross-polymers comprising (i) an effective amount of at least one saliva and moisture impermeable layer and (ii) an effective amount of at least one moisture and saliva permeable alkaline sublayer having the pH adjusted between 6.5 and 10.5 and (B) an effective amount of bleach-stable hydrophilic acid layer containing therein dispersed, dissolved, emulsed or otherwise distributed at least one peroxygen compound having the pH adjusted between about 4.5 and 6.8 layer and said multilayered system to the tooth surfaces and exposed to saliva and moisture to react to cause whitening activity.

18. A method of claim 17 wherein said at least one hydrophobic moisture impermeable layer is present in the operative range of about 2.5% and 75% with respect to the total weight of the composition and wherein at least one moisture permeable alkaline sublayer is present in the operative. range of about 2.5% and 75% with respect to the total weight of the composition and wherein an effective amount of bleach-stable hydrophilic acid layer is present in the operative range of about 2.5% and 75% with respect to the total weight of the composition.

19. The method of claim 17 wherein said bleach stable hydrophilic acid layer includes a peroxygen compound. present in said composition in an amount of about 0.5 and 55 percent by weight of the total weight of said composition and wherein said separate alkaline sublayer comprises an alkaline composition present in said composition in an amount of about 2.5 to 75 percent by weight of the total weight of said composition.

20. The method of claim 17 wherein said peroxygen compound is selected from the group consisting of hydrogen peroxide, carbamide peroxide, calcium peroxide, sodium peroxide, sodium carbonate peroxide, calcium carbonate peroxide, stabilized chloride peroxide, sodium perborate, sodium percarbonate, magnesium peroxide; peracids such as magnesium monoperoxyphthalate, diperoxydodecanedioic acid, peroxydiphosphate, sodium perborate monohydrate, sodium carbonate polyoxyhydrate, etc; enzymes such as oxyoxydoreductaze, lysozyme, and mixtures thereof; and wherein said catalyst alkaline compound is selected from the group consisting of sodium hydroxide, calcium hydroxide, ammonium hydroxide, magnesium hydroxide, sodium bicarbonate, sodium carbonate, sodium phosphate dibasic and sodium phosphate tribasic.
